(19) United States
(12) Patent Application Publication
(10) Pub. No.: US 2026/0356993 A1
(43) Pub. Date: Nov. 12, 2026

(54) A COMPOSITION AND FABRICATION OF SENSOR MATERIAL USING MOLECULAR IMPRINTED POLYMER LAYER FOR DETECTION OF VOLATILES

(71) Applicant: ACCUBITS INVENT PRIVATE LIMITED, Trivandrum (IN)

(72) Inventors: Allen J A, Trivandrum (IN); Viji S, Trivandrum (IN); Shilpa S, Trivandrum (IN); Sajini S, Trivandrum (IN); Navaneeth SUBASH, Trivandrum (IN); Nidhin SREEKUMAR, Trivandrum (IN)

(73) Assignee: ACCUBITS INVENT PRIVATE LIMITED, Trivandrum, KL (IN)

(21) Appl. No.: 19/124,001

(22) PCT Filed: Nov. 25, 2023
(86) PCT No.: PCT/IN2023/051097

§ 371 Date: Apr. 24, 2025

(30) Foreign Application Priority Data
Nov. 25, 2022 (IN) 202241064949

(51) Int. Cl. C08F 2/50; B05D 3/06; B05D 7/00; C08F 2/06; C08F 20/18; G01N 27/04; G01N 33/00

(52) U.S. Cl. CPC C08F 2/50; B05D 3/067; B05D 7/544; C08F 2/06; C08F 20/18; G01N 27/04; G01N 33/0047; B05D 2203/30; B05D 2502/00; B05D 2518/00

(57) ABSTRACT

The present invention discloses a composition and fabrication method of sensor using molecular imprinted polymer for detection of volatiles A semiconducting material, like Polyaniline coated substrates of appropriate size were taken in a mixed porogen solvent of acetonitrile (ACN) and toluene along with a monomer such as methacrylic acid (MAA). The template, monomer (MAA), and cross-linker (ethylene glycol dimethacrylate (EGDMA)) ratios are kept constant at n:4:8 respectively, wherein ‘n’ ranges about 1-5. The pre-polymer mixture is then stirred in a nitrogen atmosphere with 2,2′-azobisisobutyronitrile (AIBN), an initiator. The coated substrates were then immersed in the pre-polymer mixture and purged with nitrogen and placed in a UV chamber for photo-polymerization at room temperature. Once the polymerization reaction is completed, the template volatile organic compound is extracted with methanol and acetic acid leaving molecular cavities of the template volatile organic compound imprinted in the polymer matrix that allows the polymer to bind with the particular template molecule with high selectivity for the detection of volatiles.

### FIELD OF INVENTION

The invention relates to the field of molecular imprinting of polymers. The invention specifically relates to method of making molecularly imprinted polymer using a combination of bulk polymerization and photo-polymerization technique. The invention also relates to a composition for making molecularly imprinted polymer over a semiconducting polymer layer on a substrate for chemiresistive sensing of volatile organic compounds.

### BACKGROUND OF INVENTION

Molecularly imprinted polymers (MIP) are synthetic polymers (artificial receptors) that recognize template molecules with remarkable specificity. Monomers form a complex with a template by covalent or non-covalent interactions and are subsequently linked via a crosslinking agent in the most typical production process. Other than having antibody-like molecular selectivity, the primary advantages of MIPs over biological analogues includes physical robustness, resistance to high temperatures and pressures, inertness to acids, bases, and organic solvents, as well as low production cost and ease of synthesis. Non-covalent imprinting is the most often utilized approach for creating MIPs. Non-covalent interactions such as hydrogen bonding, electrostatic forces, van der Waals forces, or hydrophobic interactions produce the complex of the template and the functional monomer in situ during this process. In a suitable solvent, the functional monomer, template, cross-linker, and initiator are combined to form the MIP. This approach has numerous advantages, including rapid synthesis of the template-monomer complex and very easy removal of the templates from the polymers, quick binding of templates to MIPs, and application to a wide range of target molecules. However, the polymerization conditions must be carefully adjusted to reduce non-specific binding sites in order to enhance the creation of the labile complex of template and monomer.

Selection of an appropriate synthesis technique is essential for the synthesis of MIP with the requisite characteristics. Bulk polymerization, the most often used free radical polymerization, requires mechanical grinding and sieving processes to get small particles. Low yield, fewer binding sites as a result of grinding damage, and difficulties extracting the template from the inside of the MIP particles are the primary drawbacks of bulk polymerization.

Photo-polymerization is another typical method for producing MIP. Because light is directly responsible for radical generation, the temperature can be set to low values, which protects temperature-sensitive, non-covalent interactions between the template and functional monomers, improving imprinting efficiency and the MIP's affinity for its target and, in some cases, suppressing undesired side reactions. Photochemical techniques, by definition, provide for spatiotemporal and intensity control over the polymerization reaction. When compared to other processes such as thermal polymerization, radical production may be instantly switched “on” or “off” by simply turning on or off the light source. As shown in different photolithographic and 3D printing techniques, light can also be contained into small volumes to arbitrarily trigger localized polymerizations in constrained locations. Changing the wavelength and intensity of light sources, analogous to modifying the temperature in a thermal polymerization, allows for manipulation of the polymerization process by influencing the amount of produced radicals. All of these characteristics, as well as the relatively low cost and availability of a variety of light sources (e.g., lamps, LED, lasers, UV plasma sources, sunlight), make photo-polymerization a practical strategy for precise, hierarchical structuring and even automation in the design and fabrication of MIPs.

WO2011095033A1 discloses a preparation method for molecular recognition sensor by electrodeposition is provided. The preparation method includes: forming sol gel coating template molecules by self-assembly of ionic type photosensitive copolymer; forming a film on a surface of an electrode by electrodepositing at a constant potential; crosslinking the sol gel by ultraviolet light irradiation; eluting the template molecules to obtain the electrode modified by sol gel film; and connecting the modified electrode with a sensor device and a computer to assemble a molecular recognition sensor capable of specifically detecting the template molecules

U.S. Pat. No. 6,310,110B1 discloses a method of making a molecularly imprinted porous structure makes use of a surfactant analog of the molecule to be imprinted that has the imprint molecule portion serving as the surfactant headgroup. The surfactant analog is allowed to self-assemble in a mixture to create at least one supramolecular structure having exposed imprint groups. The imprinted porous structure is formed by adding reactive monomers to the mixture and allowing the monomers to polymerize, with the supramolecular structure serving as a template. The resulting solid structure has a shape that is complementary to the shape of the supramolecular structure and has cavities that are the mirror image of the imprint group. Similarly, molecularly imprinted particles may be made by using the surfactant to create a water-in-oil microemulsion wherein the imprint groups are exposed to the water phase. When reactive monomers are allowed to polymerize in the water phase to form particles, the surface of the particles have cavities that are the mirror image of the imprint group.

U.S. Pat. No. 11,193,940 B2 discloses a sensor for the detection of Neurotrophic Factor (NF). The sensor, preferably a Screen Printed Electrochemical sensor (SPE), has a working electrode coated by a Molecularly Imprinted Polymer (MIP) imprinted by a NF. The invention also relates to a method for preparing such a sensor and comprising the steps of 1) formation of a cleavable linking layer on the working electrode of the sensor; 2) immobilization of NF molecules on the cleavable linking layer; 3) polymerization of m—PD on the working electrode of the sensor; and 4) cleavage of the cleavable linking layer thereby removing the NF molecules from the MIP layer.

The present invention overcomes the disadvantages of bulk polymerization. We have developed a combination technique using bulk polymerization and photo polymerization, and produced a thin film based MIP sensor. This has various advantages such as reduction in total solvent volume, short reaction time, structural stability, easy removal of template molecule, absence of any grinding procedure and hence binding sites formed will be accurate for the template.

### SUMMARY OF INVENTION

The present invention relates to a method of preparing molecular imprinting layer over a substrate. The imprinting layer is over a conductive layer, wherein the semiconductive property enables chemiresistive sensing of volatile organic compounds.

Another objective of the invention is to provide a sensor with high sensitivity and selectivity, comprising of a molecularly imprinted polymer for sensing various volatile organic compounds.

In one aspect, the present invention provides a method of formation of molecularly imprinted polymer (MIP) layer of nanostructured thin film over a substrate.

In another embodiment of the invention, the method of formation of molecularly imprinted polymer (MIP) uses a combination of bulk polymerization and photopolymerization technique. using UV light.

In another aspect, the present invention provides a method for preparing polyaniline (PANI) conducting polymer layer over alumina substrate and utilize the substrate for deposition of MIP layer.

Using the technique disclosed in the specification, MIP thin films is made for the detection of any volatile organic compounds (VOCs), from plants, humans, microbes, pollutants, explosives, drugs etc such as alcohols, aldehydes, aromatic compounds, ketones, styrene, and cyclic ketones etc, with high sensitivity and superior selectivity.

### BRIEF DESCRIPTION OF DRAWINGS

The foregoing summary, as well as the following detailed description of the invention will be better understood when read in conjunction with the appended drawings. For the purpose of assisting in the explanation of the invention, there are shown in the drawings, embodiments which are presently preferred and considered illustrative. It should be understood, however, that the invention is not limited to the precise arrangements and instrumentalities shown therein. In the drawings:

FIG. 1 illustrates schematic diagram for the formation of molecularly imprinted polymers (MIP);

FIG. 2 illustrates plausible mechanism for the formation of MIP and removal of template in MIP for 2-Phenylethanol;

FIG. 3 illustrates SEM micrographs of 2-Phenylethanol MIP coated PANI samples before (a-b) and after (c-d) extraction process;

FIG. 4 illustrates a) Sensitivity of 2-Phenylethanol for various concentration range, b) Response of 2-Phenylethanol, c) Response vs recovery graph for 2-Phenylethanol, d) Sensitivity of 2-Phenylethanol;

FIG. 5 illustrates a) Selectivity of 2-Phenylethanol with other volatiles, b) Transient studies of 2-Phenylethanol response;

FIG. 6 illustrates plausible mechanism for the formation of MIP and removal of template in MIP for Heptanal;

FIG. 7 illustrates FESEM micrographs of Heptanal MIP coated PANI samples before (a-b) and after (c-d) extraction process;

FIG. 8 illustrates a) Sensitivity of Heptanal for various concentration range, b) Response of Heptanal, c) Response vs recovery graph for Heptanal, d) Sensitivity of Heptanal;

FIG. 9 illustrates a) Selectivity of Heptanal with other volatiles, b) Transient studies of Heptanal response;

FIG. 10 illustrates plausible mechanism for the formation of MIP and removal of template in MIP for Toluene;

FIG. 11 illustrates FESEM micrographs of Toluene MIP coated PANI samples before (a-b) and after (c-d) extraction process;

FIG. 12 illustrates a) Sensitivity of Toluene for various concentration range, b) Response of Toluene, c) Response vs recovery graph for Toluene, d) Sensitivity of Toluene;

FIG. 13 illustrates a) Selectivity of Toluene with other volatiles, b) Transient studies of Toluene response;

FIG. 14 illustrates plausible mechanism for the formation of MIP and removal of template in MIP for 4-Vinylanisole;

FIG. 15 illustrates FESEM micrographs of 4-Vinylanisole MIP coated PANI samples before (a-b) and after (c-d) extraction process;

FIG. 16 illustrates a) Sensitivity of 4-Vinylanisole for various concentration range, b) Response of 4-Vinylanisole, c) Response vs recovery graph for 4-Vinylanisole, d) Sensitivity of 4-Vinylanisole;

FIG. 17 illustrates a) Selectivity of 4-Vinylanisole with other volatiles, b) Transient studies of 4-Vinylanisole response;

FIG. 18 illustrates plausible mechanism for the formation of MIP and removal of template in MIP for 2-Butanone;

FIG. 19 illustrates FESEM micrographs of 2-Butanone MIP coated PANI samples before (a-b) and after (c-d) extraction process;

FIG. 20 illustrates a) Sensitivity of 2-Butanone for various concentration range, b) Response of 2-Butanone, c) Response vs recovery graph for 2-Butanone, d) Sensitivity of 2-Butanone;

FIG. 21 illustrates a) Selectivity of 2-Butanone with other volatiles, b) Transient studies of 2-Butanone response;

FIG. 22 illustrates plausible mechanism for the formation of MIP and removal of template in MIP for Cyclohexanone;

FIG. 23 illustrates FESEM micrographs of Cyclohexanone MIP coated PANI samples before (a-b) and after (c-d) extraction process;

FIG. 24 illustrates a) Sensitivity of Cyclohexanone for various concentration range, b) Response of Cyclohexanone, c) Response vs recovery graph for Cyclohexanone d) Sensitivity of Cyclohexanone;

FIG. 25 illustrates a) Selectivity of Cyclohexanone with other volatiles, b) Transient studies of Cyclohexanone response.

### DETAILED DESCRIPTION OF INVENTION

The present invention will now be described more fully herein after. For the purposes of the following detailed description, it is to be understood that the invention may assume various alternative variations and step sequences, except where expressly specified to the contrary. Thus, before describing the present invention in detail, it is to be understood that this invention is not limited to particularly exemplified systems or embodiments that may of course, vary. The use of examples anywhere in this specification including examples of any terms discussed herein is illustrative only, and in no way limits the scope and meaning of the invention or of any exemplified term. Likewise, the invention is not limited to various embodiments given in this specification.

As used herein, the singular forms “a,” “an,” and “the” include plural reference unless the context clearly dictates otherwise. The term “and/or” means one or all of the listed elements or a combination of any two or more of the listed elements.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

When the term “about” is used in describing a value or an endpoint of a range, the disclosure should be understood to include both the specific value and endpoint referred to.

As used herein the terms “comprises”, “comprising”, “includes”, “including”, “containing”, “characterized by”, “having” or any other variation thereof, are intended to cover a non-exclusive inclusion.

The term ‘substrate’ used herein refers to a base material on to which a chemical coating may be provided. The substrate may be a ceramic, alumina, metals, glass slides, fabrics, fluorine doped tin oxide substrates, indium doped tin oxide substrates, silicon wafers, flexible polymers etc.

The term ‘template’ used herein refers to the volatile organic compound used for the formation of molecularly imprinted polymer and their corresponding sensing.

The term ‘sensor’ herein refers to the molecularly imprinted polymer layer formed over a substrate, the layer having conductive properties. The substrate has two gold metal contacts for measuring resistance change using a digital multimeter when a template volatile organic compound interacts with the surface of the molecularly imprinted polymer.

The term ‘molecular imprinting’ used herein refers to a technique of creating artificial recognition sites in polymeric matrices which are complementary to the template in their size, shape and spatial arrangement of the functional groups.

The term ‘molecularly imprinted polymers’ used herein refers to synthetic polymers that are artificial receptors, that recognize template molecules with remarkable specificity.

The term non-imprinted polymer used herein refers to synthetic polymers without artificial receptors that cannot recognize template molecules with remarkable specificity.

The term ‘combination of bulk and photo polymerization’ used herein refers to the method used in the formation of molecularly imprinted polymer (MIP) and non-imprinted polymer (NIP). Herein the polymerization method such as electropolymerization, microwave polymerization, precipitation polymerization, Reversible addition-fragmentation chain-transfer (RAFT) polymerization, bulk polymerization, surface imprinting, lithography imprinting, immobilization imprinting, micro contact imprinting, and emulsion polymerization can also be used for the preparation of MIP and NIP.

The term ‘conducting layer’ and ‘conductive layer’ used herein refers to a semiconducting layer which conduct electricity or heat through it. The said layer has semiconducting nature and can measure resistance change while interacting with volatile organic compounds. The semiconducting layer used herein may be selected from polyaniline, polypyrrole, poly(3,4-ethylenedioxythiophene) polystyrene sulfonate (PEDOT:PSS), polythiopene, polyacetylene etc or carbon nanotubes, activated carbon, graphene oxide, reduced graphene oxide, class of MXene materials, siloxene, phosphorene, black phosphorus, germanene and graphitic carbon nitride, metal oxides, metal chalcogenides, metal phosphides, metal vanadates, etc, their combinations of composite materials and alloys thereof. These organic polymers have unique electrical and optical properties similar to those of inorganic semiconductors. Therefore, it is mentioned as conducting layer.

The term ‘piranha treatment’ used herein refers to treatment of substrates using piranha solutions. Piranha solutions are a mixture of concentrated sulfuric acid with hydrogen peroxide, usually in a ratio of 3:1 to 7:1. They are used to remove trace amounts of organic residues, such as photoresist, from substrates.

The term ‘porogen solvent’ used herein refers to the solvents used in the preparation of molecularly imprinted polymers. The porogen solvent is a mixture of acetonitrile (ACN) and toluene in a volume of 6.75 mL and 0.75 mL. The porogen solvent also includes 2-methoxyethanol, methanol, chloroform, tetrahydrofuran (THF), dichloroethane, N,N-dimethylformamide (DMF) and ionic liquids such as 1-Butyl-3 methylimidazolium hexafluorophosphate [BMIM][PF6], 1-Butyl-3 methylimidazolium tetrafluoroborate [BMIM][BF4], 1-Methyl-3-octyl-imidazolium-hexafluorophosphate [OMIM][PF6] and 1-Hexyl-3-methyl-imidazolium-hexafluorophosphate [HMIM][PF6] etc.

The following detailed description discloses the technical features of the invention and its operation fully along with examples.

The present invention relates to a method of preparing molecularly imprinted polymers. A layer of polymer having conductive property is deposited over a substrate. An imprinting polymer layer is over the conducting layer, wherein the conductive property enables chemiresistive sensing of volatile organic compounds. Thus, the method also relates to preparation of a sensor for sensing volatile organic compounds.

Preparation of polyaniline (PANI) films: In an embodiment of the invention a substate is coated with a conductive layer. In an example polyaniline is used as the conducting layer, which is coated over a ceramic alumina substrate. The semiconducting layer can be selected from polyaniline, carbon nantotubes, activated carbon, graphene oxide, reduced graphene oxide, class of MXene materials, siloxene, phosphorene, black phosphorus, germanene and graphitic carbon nitride, metal oxides, metal chalcogenides, metal phosphides, metal vanadates etc, their combinations of composite materials and alloys thereof.

The polyaniline (PANI) coated alumina substrates are synthesized by means of oxidative polymerization using modified successive ionic layer adsorption and reaction (SILAR) method. Polyaniline is a promising and most researched conducting polymer which exhibits great stability, processability, tuneable conductivity, and optical properties. Polyaniline conductivity is proportional to dopant concentration, and it exhibits metal-like conductivity only when the pH is less than 3. Polyaniline becomes conductive, after being moderately oxidized and behaves as an insulator when fully oxidized.

The process involves preparation of a cationic precursor solution maintained at room temperature and an anionic precursor solution maintained at 4 °C temperature in an ice bath. The cationic precursor is made up of 0.1M aniline in 0.1M of sulphuric acid and the anionic precursor is made up of 0.1M ammonium persulphate in 0.1M of sulphuric acid. The alumina substrate undergoes a piranha treatment and cleaning process for uniform deposition. The alumina substrate is first immersed in the cationic precursor for 20 sec which initiates the absorption of anilinium cations on the surface of the alumina substrate. Then it is removed and immersed in the anionic precursor kept in an ice bath for 15 sec. This process is continued for 50 cycles. Here the oxidation of aniline caused due to its reaction with ammonium persulphate (APS) is formed leading to the formation of PANI film. The oxidation process leaves behind a thick emeraldine green layer of PANI on the alumina substrate. After 50 cycles the PANI deposited alumina substrates were immersed in double distilled water to remove the unbound and unreacted anilinium ions and dried in vacuum oven at about 80-90 °C, preferably 85 °C for 24 hours. The polyaniline (PANI) coated alumina substrates are thus synthesized.

Fabrication of imprinting layer over PANI coated ceramic substrate: FIG. 1 shows schematic diagram for the formation of molecularly imprinted polymer (MIP) for different volatiles as templates. The polyaniline coated alumina ceramic substrates were taken in the appropriate size for preparing molecularly imprinted polymer and non-imprinted polymer. For the polymerization, a mixed porogen solvent of acetonitrile (ACN) and toluene is used. The template, monomer, and cross-linker ratios are kept constant at n:4:8, wherein ‘n’ is a value between 1-5. The distribution of the molecularly imprinted polymer over semiconducting layer is in the form of a continuous layer with template-voids, wherein the entire layer is electrically conductive.

A porogen solvent is prepared containing acetonitrile and toluene in a ratio of 6.75 mL and 0.75 mL. The porogen solvent is taken in a glass container such as a glass vial and sonicated for 60 seconds. To the solvent a template and a monomer is added and ultrasonicated for 15 minutes. To this mixture a cross-linking agent is added and purged with nitrogen for about 120 seconds to remove the oxygen in the vials before being subjecting to ultra sonication for 10 minutes, followed by stirring for 10 minutes. A prepolymer mixture is obtained. The prepolymer mixture is then stirred for 15 minutes in a nitrogen atmosphere with a photo initiator. The PANI coated alumina substrates are then immersed in the prepolymer mixture and purged with nitrogen for about 2 minutes before being carefully sealing with a screw cover to prevent leakage and oxidation. The screw cap vial is placed in a UV chamber equipped with 8 W UV bulbs with wavelength 2 of 350 nm and photopolymerized at room temperature. The PANI coated alumina substrates were removed after the specified time and left to dry in normal atmospheric conditions. When exposed to atmospheric conditions, a thick white layer of molecularly imprinted polymer layer forms on the surface of PANI in just as few seconds. In the absence of a template, a similar process is used to create a non-imprinting polymer layer (NIP). The template molecule is removed after synthesis to investigate the rebinding capacity. The MIP-PANI substrates were immersed in a mixture of about 20-50 μL acetic acid and about 200 mL methanol solution and extracted for about 12 hours using a soxhlet apparatus at 80 to 90 °C, preferably at 85 °C. After the extraction process the samples were stored in a vacuum desiccator at atmospheric temperature of about 27 °C.

In an embodiment the monomer can be selected from methacrylic acid, acrylamide, 4-Vinylpyridine, acrylic acid, 2-Hydroxyethyl Methacrylate (HEMA), styrene, N,N′-Methylenebisacrylamide (BIS), N-Methacryloyl-(L)-phenylalanine (MAPA), N-Isopropylacrylamide (NIPAM), divinylbenzene (DVB). In an embodiment, the volatile organic compound can be selected from alcohol, aldehyde, aromatic compounds, styrene, ketone, cyclic ketone, esters, amines, organochlorides, organophosphates, alkanes, alkyne and alkenes.

In another embodiment, the crosslinker can be selected from ethylene glycol dimethacrylate, trimethylolpropane trimethacrylate (TRIM), pentaerythritol trimethacrylate (PETA), N,N′-Methylenebisacrylamide (BIS), 1,4-Butanediol Dimethacrylate (BDDMA), 1,2-Bis(allyloxy)ethane (BAE).

In yet another embodiment, the initiator can be selected from 2,2′-azobisisobutyronitrile (AIBN), 2,2-dimethoxy-2-phenylacetophenone (DMPA), azobis (2-methylpropionitrile) (AMBN), Benzoyl peroxide, 2,2′-azobis(2,4-dimethylvaleronitrile) (ADMVN), N,N,N′,N′-tetramethylethylenediamine (TEMED).

Deposition of gold metal contacts on imprinted polymer layer coated PANI substrates: Molecularly imprinted polymer coated polyaniline (MIP-PANI) and non-imprinting polymer coated polyaniline substrates (NIP-PANI) were prepared for the sensing of volatile organic compounds. Therefore, a thin interdigitated electrode (IDE) layer is coated over MIP-PANI and NIP-PANI. The deposition of gold IDE contacts over the MIP coated PANI and NIP coated PANI substrates were performed.

Preparation of a sensor: A sensor was prepared from MIP for sensing volatile organic compounds. The sensor comprises at least two electrodes, and a molecularly imprinted polymer over a semiconducting layer coated substrate as sensing element which is electrically conductive.

Sensing of VOCs using MIP coated PANI and NIP coated PANI substrates: MIP after extraction and NIP after extraction samples were subjected to sensing different VOCs. Initial stabilization of the sensor in dry air is set for about 30 min. After that corresponding volatile is purged at part per million levels to gain the response of the sensor. MIP sensor responded on increasing the concentration of volatiles. Interference studies were performed with volatiles with similar and dissimilar chemical structure and vapour pressure. This proves the high selectivity and sensitivity of MIP for corresponding VOCs.

The prepared molecularly imprinted polymer sensor are used to detect their respective volatile organic compound. Using a specialized bubbler arrangement, the volatile organic compound (VOC) is purged for measuring the response. Prior to purging VOC, dry air is injected, followed by purging with a fixed amount of VOC for sensing.

### EXAMPLES

### Example 1-Preparation of MIP and NIP for Alcohols (Eg Alcohol-2-Phenylethanol)

2-Phenylethanol (2-PE) is a significant plant volatile component formed from phenylalanine that contributes significantly to the flavor of many meals, notably fresh fruits like tomatoes as well as the fragrance of many flowers, including roses, that attracts insects. A key role for 2-phenylethanol is played during tomato fruit ripening, when the color of the fruit changes from green to red as a result of the conversion of chloroplasts to chromoplasts.

Polyaniline (PANI) coated alumina substrates were taken in the size of about 1 cm² for this application. For polymerization step, a mixed porogen solvent of acetonitrile (ACN) and toluene is used. The template, monomer, and cross-linker ratios are kept constant at 1:4:8 respectively. The porogen solvent is sonicated for 30 to 90 seconds in volumes of acetonitrile (ACN) 5 to 8 mL and toluene 0.50 to 1.0 mL, respectively. The porogenic solvent combination is then ultrasonicated for about 15 minutes with 7-12 μL of 2-Phenylethanol (2-PE) as a template and 20 to 30 μL methacrylic acid (MAA) as a monomer. To this mixture ethylene glycol dimethacrylate (EGDMA) cross-linker is added in a volume of 100-120 μL and purged with nitrogen for about 120 seconds to remove the oxygen in the vials before being subjected to ultrasonication for about 10 minutes and followed by stirring for about 10 minutes. The pre-polymer mixture is then stirred for about 15 minutes in a nitrogen atmosphere with 10.0 to 15.0 mg of 2,2′-azobisisobutyronitrile (AIBN), a initiator. The PANI-coated alumina substrates are then immersed in the pre-polymer mixture and purged with nitrogen for about 120 to 200 seconds before carefully sealing with a screw cap to prevent leakage and to prevent mixing of the contents with oxygen. The screw cap vial is placed in a UV chamber equipped with 8 W UV bulbs with wavelength of 300 to 400 nm and photopolymerized for about 60-100 minutes at room temperature. The PANI-coated alumina substrates were removed after about 6-10 minutes and left to dry in atmospheric conditions at room temperature. When exposed to atmospheric conditions, a thick white layer of molecularly imprinted polymer layer forms on the surface of PANI in seconds. To create a non-imprinting polymer layer (NIP) in the absence of a template, the same process is followed.

The template molecule is removed after synthesis to investigate the rebinding capacity using an extraction process. As a result, the MIP-PANI substrates were immersed in a mixture of about 20-50 μL acetic acid and about 150-200 mL methanol solution and extracted for about 12 hours using a soxhlet apparatus at 80 to 90 °C, preferably at 85 °C, for the removal of template volatile organic compounds and creation of template-voids. After the extraction process, the samples were stored in a vacuum desiccator.

### Example 2: Plausible Mechanism for MIP Formation for 2-Phenylethanol

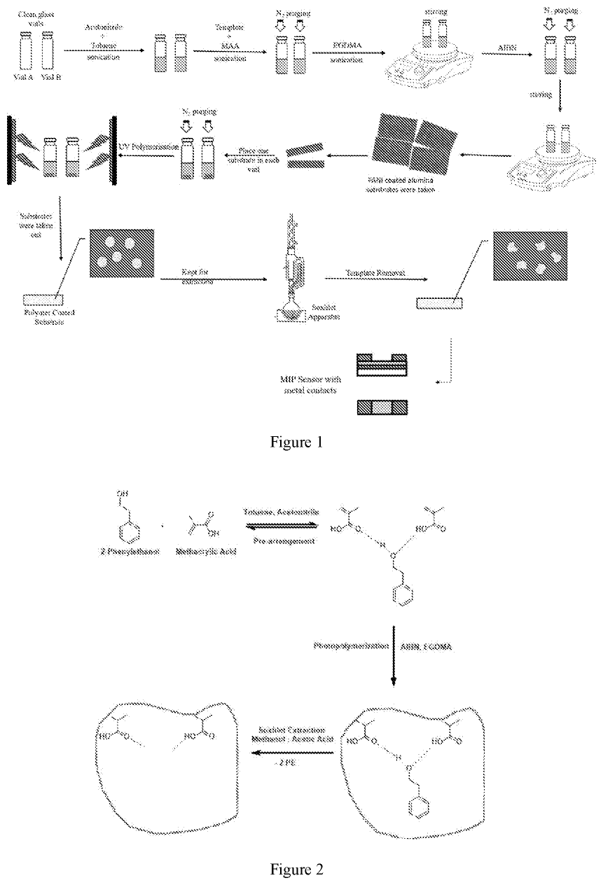

The imprinting process begins with a combination between a functional monomer and a template that involves hydrogen bonding. Here, the methacrylic acid (MAA) is used as a hydrogen bonding monomer because the carboxyl group can act as a hydrogen donor as well as an acceptor at the same time. Non-polar solvents such as toluene and ACN are utilized in order to promote the interaction between template and functional monomer. MAA consists of two units, one is a recognition unit and the other is a polymerization unit. The mechanism and interaction between a functional monomer and template molecule (2-Phenyl ethanol) occur in the pre-polymerization process. By adding ethylene glycol dimethacrylate (EGDMA), a cross-linker, followed by 2,2′-Azobis(2-methylpropionitrile) (AIBN) a photo-initiator, to the pre-polymer complex, free radical polymerization reaction begins upon UV illumination. EGDMA works as a cross-linker molecule to link the template and functional monomer complex into a single continuous polymer matrix upon photo-initiation. AIBN decomposes in the presence of UV illumination to form a 2-cyanopropyl radical with the elimination of one molecule of nitrogen. These 2-cyanopropyl fragments with unpaired electrons are the free radical initiators. Following these continuous reactions, they react with the polymerization unit in the pre-polymer complex thereby creating a growing polymer chain. Once the polymerization reaction is completed, the template molecule is extracted with methanol and acetic acid leaving molecular cavities of the template molecule imprinted in the polymer matrix that allows the polymer to bind with the particular template molecule with high selectivity. Schematic representation of formation of molecularly imprinted polymers is provided in FIG. 1 and mechanism for the formation of MIP, NIP and removal of template in MIP is provided in FIG. 2.

### Example 3: SEM Analysis of 2-Phenylethanol-MIP

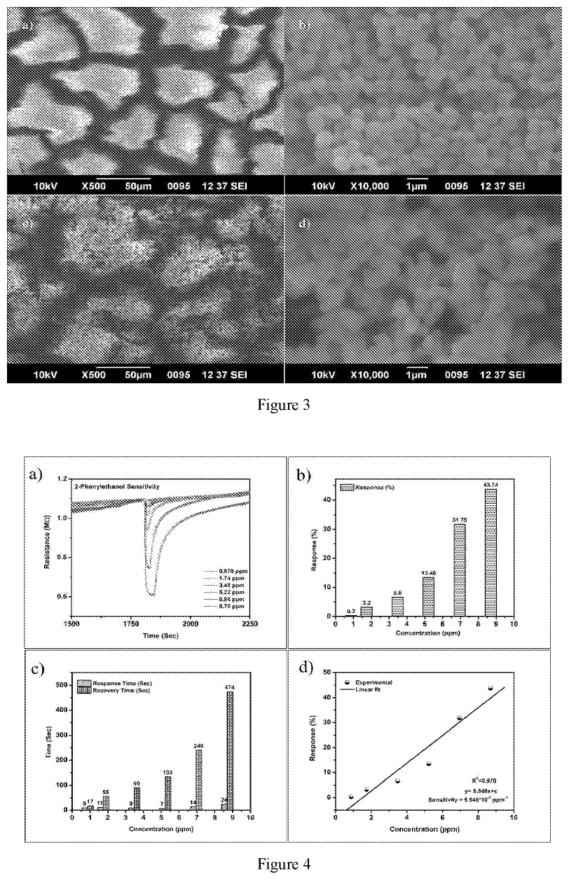

The Scanning Electron Microscope (SEM) micrographs of MIP coated PANI substrates (FIG. 3) before and after extraction reveals a change in their surface morphology. In FIG. a) the macro porous structure of the MIP layer is visible as cracks at a 50 μm scale. When it is further magnified, uniform spherical polymeric nanoparticles are visible at a 1 μm scale as in FIG. 3 b) on 2-Phenylethanol MIP before extraction. Similar to the FIG. 3 a) and b) the “after extraction sample” is found to have cracks with macro porous structure along with visible micro pores formed as voids after the removal of the template molecule as in FIG. 3 c). In a similar magnification of 1 μm scale as in FIG. 3 d) the spherical morphology found in “before extraction sample” is changed into irregular agglomerated particles.

### Example 4: Sensitivity of 2-Phenylethanol MIP Sensor

The response of 2-Phenylethanol to the MIP sensor were studied using their VOC chemiresistive sensing. The FIG. 4 a) displays the sensitivity of the MIP sensor for 2-Phenylethanol vapor maintained in an ice bath temperature at 4 °C with concentrations ranging from 878 ppb to 8.7 ppm. In FIG. 4 b) the response R (%) is calculated to obtain the response of the 2-Phenylethanol MIP sensor and it is calculated to be 0.3, 3.2, 6.6, 13.46, 31.75 and 43.74% for 878 ppb, 1.74 ppm, 3.48 ppm, 5.22 ppm, 6.96 ppm, and 8.70 ppm. The sensor's high speed is confirmed by the typical response time of 9, 11, 9, 7, 14 and 24 is observed with corresponding recovery time of 17, 55, 90, 133, 240, and 474 seconds for 878 ppb, 1.74 ppm, 3.48 ppm, 5.22 ppm, 6.96 ppm, and 8.70 ppm as in FIG. 4 c). This explains the swiftness of the sensor in the low concentration as it responds faster. When the concentration is high the recovery time increases due to the unbinding of 2-Phenylethanol VOC from the binding sites of MIP. During sensing, the 2-Phenylethanol VOC binds with the active voids created due to the extraction, as a result of which a change in resistance value is observed with the corresponding concentration of the 2-Phenylethanol. We will be able to identify the traces of 2-Phenylethanol vapor in actual surroundings and precisely assess the lower amounts. The 2-Phenylethanol MIP sensor exhibits a linear behaviour and exhibits a linear fit of R²=0.970 as shown in FIG. 4 d). The sensitivity of the 2-Phenylethanol sensor is measured to be 5.5*10⁻² ppm⁻¹. The theoretical lowest detection limit (LDL) of 2-Phenylethanol sensor is calculated to be 9.5 ppb by measuring 600 points from the resistance base line.

### Example 5: Selectivity of 2-Phenylethanol MIP Sensor

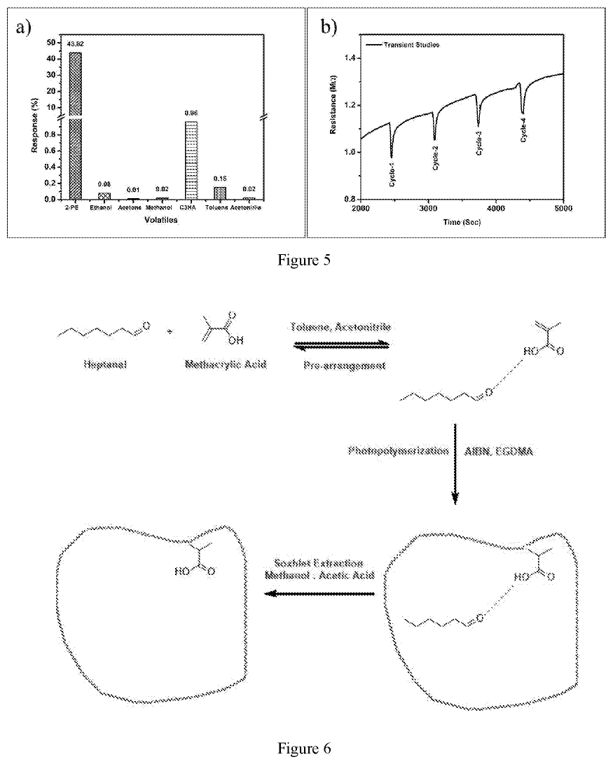

By subjecting it to the other interfering gases, the selectivity of the MIP sensor to 2-Phenylethanol was studied. When exposed to 2-Phenylethanol, ethanol, acetone, methanol, cis-3-hexenyl acetate, toluene and acetonitrile, we assessed the sensor's reaction. The response values of 2-Phenylethanol and other interfering gases is found to be 43.82, 0.08, 0.01, 0.02, 0.96, 0.15, and 0.02 %. It can be observed that the sensor's response to all the interfering gases is negligible compared to that of 5 ppm of 2-Phenylethanol, as shown in the FIG. 5 a) and it depicts the selective behaviour of the sensor to 2-Phenylethanol.

### Example 6: Transient Studies of 2-Phenylethanol MIP Sensor

By subjecting it to same concentration of 3.48 ppm, the 2-Phenylethanol MIP sensors were studied for its response and recovery for various cycles and found to exhibit same response level up to 4 cycles proving its repeatability and reproducibility of 2-Phenylethanol MIP sensor as shown in FIG. 5 b).

### Example 7: Imprinting Factor of 2-Phenylethanol MIP Sensor

The imprinting factor is the ratio of the response R(%) of the 2-Phenylethanol MIP and NIP and it is calculated to be 1.36. This proves that the 2-Phenylethanol VOC is imprinted perfectly during the polymerization reaction.

With its high sensitivity, the current 2-Phenylethanol MIP sensor offers significant potential for monitoring low quantities of 2-Phenylethanol in the environment. This 2-Phenylethanol MIP sensor marks as a significant step forward in the development of essential sensing materials for real-time volatile monitoring.

In an embodiment the alcohol can be selected from 2-phenylethanol, hexenol, linalool, nerolidol, ethanol, methanol, pinocarveol, isopropyl alcohol, pentanol etc.

### Example 8: Preparation of MIP and NIP for Aldehyde (Eg Heptanal)

Heptanal is an n-alkanal formed when the alcohol-containing hydroxy group of heptan-1-ol is oxidized to the associated aldehyde. A possible lung cancer biomarker, this endogenous aldehyde is present in the blood of patients with lung cancer and results from membrane lipid oxidation. Heptanal performs a biomarker function.

### Example 9: Preparation of Heptanal MIP and NIP

The steps are as detailed in Example 1, changes if any are detailed here. The porogenic solvent combination is ultrasonicated for about 15 minutes with 9-13 μL of heptanal as a template.

### Example 10: Plausible Mechanism for Heptanal MIP Formation

The basic mechanism is detailed in Example 2. The mechanism and interaction between a functional monomer and template molecule (Heptanal) occur in the pre-polymerization process. Heptanal is a relatively neutral aldehyde and its proton cannot be involved in hydrogen bonding with any electron donor. The carbonyl oxygen of heptanal can however form hydrogen bond complex with the acidic carboxylic proton of MAA. Schematic representation of formation of molecularly imprinted polymers is provided in FIG. 1 and mechanism for the formation of MIP, NIP and removal of template in MIP is provided in FIG. 6.

### Example 11: FESEM Analysis of Heptanal MIP

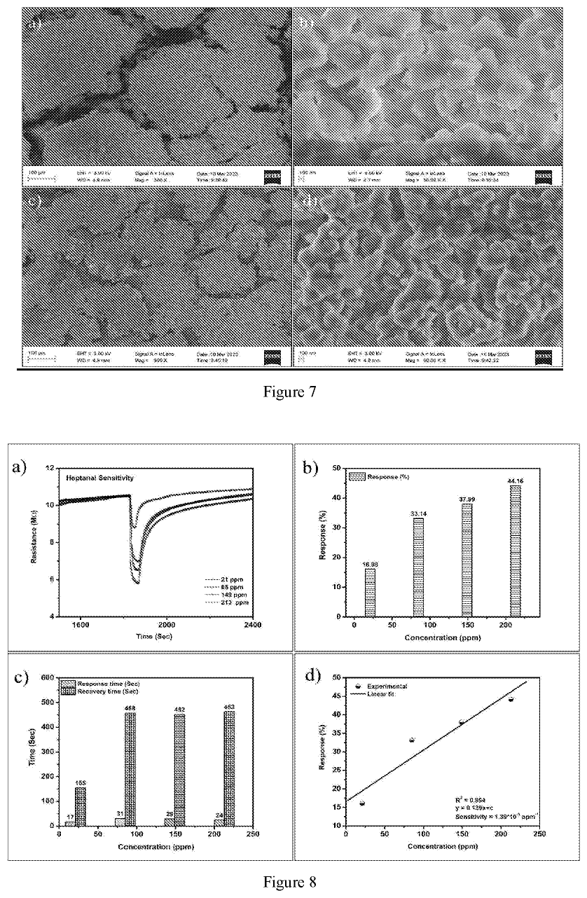

The Field Emission Scanning Electron Microscope (FESEM) micrographs of MIP coated PANI substrates (FIG. 7) before and after extraction reveals a change in their surface morphology. In FIG. 7 a) the macro porous structure of the MIP layer is visible as cracks at a 100 μm scale. When it is further magnified, non-uniform irregular polymeric nanoparticles are visible at a 100 nm scale as in FIG. 7 b) on Heptanal MIP before extraction. Similar to the FIG. 7 a) and b), the “after extraction sample” is found to have cracks with macro porous structure along with visible micro pores formed as voids after the removal of the template molecule as in FIG. 7 c). In a similar magnification of 100 nm scale as in FIG. 7 d) the morphology found “before extraction sample” is changed into irregular crumbled agglomerated particles with a decrease in diameter.

### Example 12: Sensitivity of Heptanal MIP Sensor

The response of Heptanal to the MIP sensor were studied using their VOC chemiresistive sensing. The FIG. 8 a) displays the sensitivity of the MIP sensor for Heptanal vapor maintained in an ice bath temperature at 4 °C with concentrations ranging from 21 ppm to 213 ppm. In FIG. 8 b) the response R(%) is calculated to obtain the response of the Heptanal MIP sensor and it is calculated to be 16.08, 33.14, 37.99 and 44.16% for 21 ppm, 85 ppm, 149 ppm and 213 ppm. The sensor's high speed is confirmed by the typical response time of 17, 31, 29 and 24 is observed with corresponding recovery time of 155, 458, 452, and 463 seconds for 21 ppm, 85 ppm, 149 ppm and 213 ppm as in FIG. 8 c). This explains the swiftness of the sensor in the low concentration as it responds faster. When the concentration is high the recovery time increases due to the unbinding of Heptanal molecule from the binding sites of MIP. During sensing, the Heptanal VOC binds with the active voids created after the extraction process where the template VOC is removed as a result of which a change in resistance value is observed with the corresponding concentration of the Heptanal. We will be able to identify the traces of Heptanal vapor in actual surroundings and precisely assess the low amounts. The Heptanal MIP sensor exhibits a linear behaviour and exhibits a linear fit of R²=0.954 as shown in FIG. 8 d). The sensitivity of the Heptanal sensor is measured to be 1.39*10⁻³. The theoretical lowest detection limit (LDL) of Heptanal sensor is calculated to be 274 ppb by measuring 200 points from the resistance base line.

### Example 13: Selectivity of Heptanal MIP Sensor

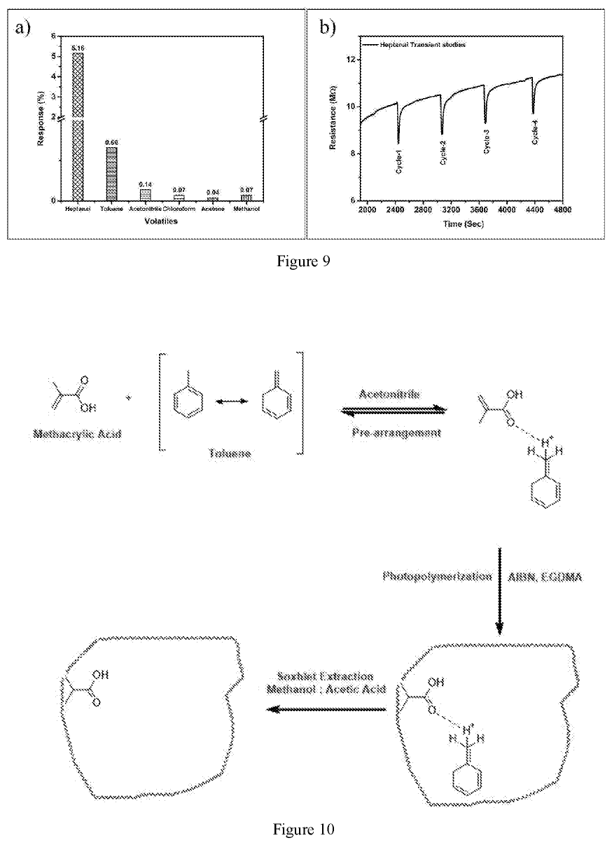

By subjecting it to the other interfering gases, the selectivity of the MIP sensor to Heptanal was studied. When exposed to Heptanal, toluene, acetonitrile, chloroform, acetone, and methanol, we assessed the sensor's reaction. The response values of Heptanal and other interfering gases is found to be 5.16, 0.66, 0.14, 0.07, 0.04 and 0.07 %. It can be observed that the sensor's response to all the interfering gases is negligible compared to that of 10 ppm of Heptanal, as shown in the FIG. 9 a) and it depicts the selective behaviour of the sensor to Heptanal.

### Example 14: Transient Studies of Heptanal MIP Sensor

By subjecting it to same concentration of 149 ppm, the Heptanal MIP sensors were studied for its response and recovery for various cycles and found to exhibit same response level up to 4 cycle proving its repeatability and reproducibility of Heptanal MIP sensor as shown in FIG. 9 b).

### Example 15: Imprinting Factor of Heptanal MIP Sensor

The imprinting factor is the ratio of the response R(%) of the Heptanal MIP and NIP and it is calculated to be 1.24. This proves that the Heptanal VOC is imprinted perfectly during the polymerization reaction.

With its high sensitivity, the current Heptanal MIP sensor offers significant potential for monitoring low quantities of Heptanal in the biological systems. This Heptanal MIP sensor marks as a significant step forward in the development of essential sensing materials for real-time volatile monitoring.

In an embodiment the aldehyde can be selected from heptanal, nonanal, pentanal, hexanal, acetaldehyde, formaldehyde, butanal, octanal, octenal, benzaldehyde, phenylacetaldehyde, cinnamaldehyde, cuminaldehyde, (E,E)-α-farnesene-2(3),9(10)-diepoxide, malondialdehyde, hydroxyacetalydehyde, 2-butenal, dimethylbenzaldehyde, phenylpropionaldehyde, perillic aldehyde, methional, hexenal, pentenal etc.

### Example 16: Preparation of MIP and NIP for Aromatic Compounds (Eg Toluene)

Toluene is a typical organic substance, sometimes referred by its IUPAC systematic name, methylbenzene. Toluene is a colorless, odorless liquid that is insoluble in water. It is a volatile organic compound (VOC) and one of the aromatic hydrocarbons. Toluene can be thought of as a probe molecule that can distinguish between various tuberculosis (TB) states. It has been noted that people with various respiratory illnesses, including TB, have higher levels of toluene in their blood and breath. Toluene is a volatile chemical molecule that is frequently present in gasoline and industrial solvents. However, the human body can also manufacture it as a result of metabolic activities.

### Example 17: Preparation of Toluene MIP and NIP

The steps are as detailed in Example 1, changes if any are detailed here. The porogenic solvent is then ultrasonicated for about 15 minutes with 6-10 μL of toluene as a template.

### Example 18: Plausible Mechanism for Toluene MIP Formation

The basic mechanism is detailed in Example 2. The mechanism and interaction between a functional monomer and template molecule (Toluene) occur in the pre-polymerization process. Methyl protons of toluene are involved in a hyperconjugation. This makes the methyl proton of toluene weakly acidic and participate in hydrogen bonding interaction with MAA. Schematic representation of formation of molecularly imprinted polymers is provided in FIG. 1 and mechanism for the formation of MIP, NIP and removal of template in MIP is provided in FIG. 10.

### Example 19: FESEM Analysis of Toluene MIP

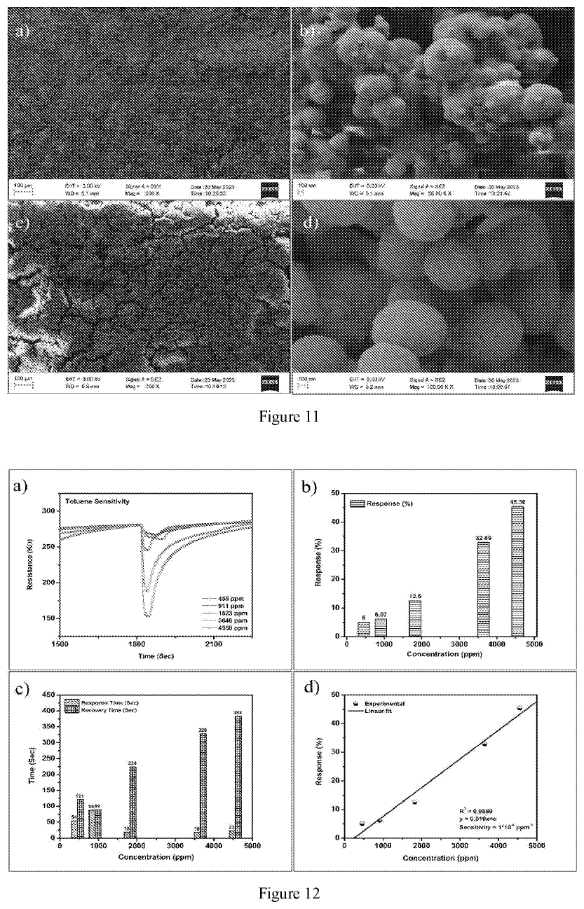

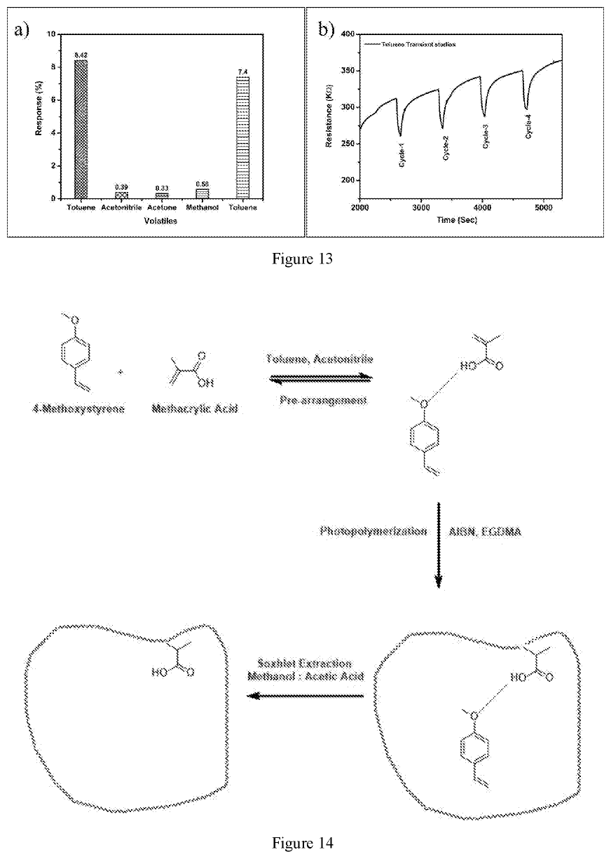

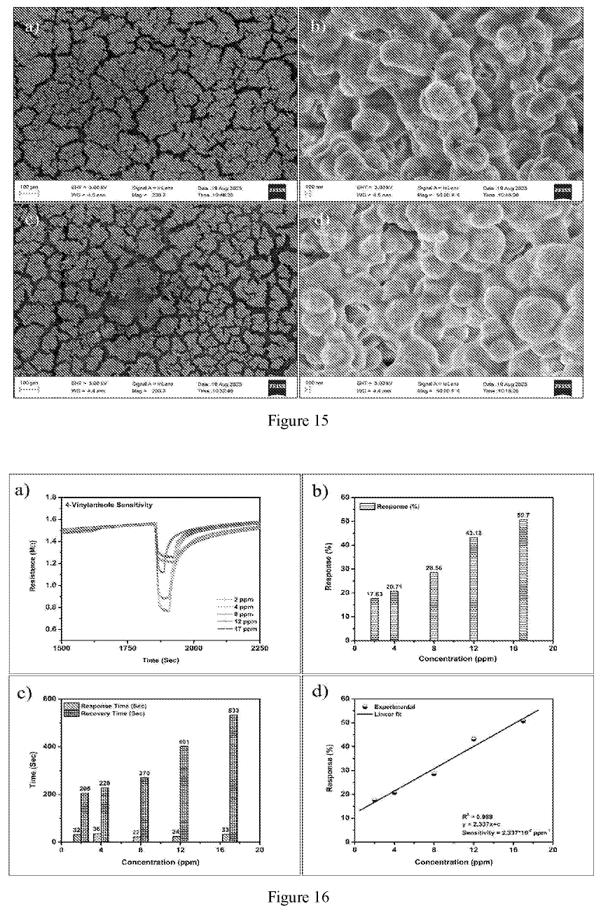

The FESEM micrographs of MIP coated PANI substrates (FIG. 11) before and after extraction reveals a change in their surface morphology. In FIG. 11 a), a surface without any cracks or pores is observed on the MIP before extraction sample at a 100 μm scale. When it is further magnified, aggregated uneven spherical polymeric nanoparticles are visible at a 100 nm scale as in FIG. 11 b) on Toluene MIP before extraction. Similar to the FIG. 13 a) and b) the “after extraction sample” is found to have cracks with macro porous structure along with visible micro pores formed as voids after the removal of the template molecule as in FIG. 11 c). In a similar magnification of 100 nm scale as in FIG. 11 d) a uniform spherical morphology is found in the “after extraction sample” MIP sample and the smaller spheres were removed during the extraction process. The surface of the spheres are found to be rough this proves the removal of template molecule toluene after the extraction process.

### Example 20: Sensitivity of Toluene MIP Sensor

The response of Toluene to the MIP sensor were studied using their VOC chemiresistive sensing. The FIG. 12 a) displays the sensitivity of the MIP sensor for Toluene vapor maintained in an ice bath temperature at 4 °C with concentrations ranging from 455 ppm to 4558 ppm. In FIG. 12 b), the response R(%) is calculated to obtain the response of the Toluene MIP sensor and it is calculated to be 5, 6.07, 12.5, 32.86, 45.36% for 455 ppm, 911 ppm, 1823 ppm, 3646 ppm, and 4558 ppm. The sensor's high speed is confirmed by the typical response time of 54, 88, 19, 18, and 23 is observed with corresponding recovery time of 121, 89, 224, 328 and 383seconds for 455 ppm, 911 ppm, 1823 ppm, 3646 ppm, and 4558 ppm as in FIG. 12 c). This explains the swiftness of the sensor in the low concentration as it responds faster. When the concentration is high the recovery time increases due to the unbinding of Toluene VOC from the binding sites of MIP. During sensing, the Toluene VOC binds with the active voids created due to the extraction, as a result of which a change in resistance value is observed with the corresponding concentration of the Toluene. We will be able to identify the traces of Toluene vapor in actual surroundings and precisely assess the low amounts. The Toluene MIP sensor exhibits a linear behaviour and exhibits a linear fit of R²=0.9899 as shown in FIG. 12 d). The sensitivity of the Toluene sensor is measured to be 1*10⁻⁴ ppm⁻¹. The theoretical lowest detection limit (LDL) of Toluene sensor is calculated to be 113 ppm by measuring 100 points from the resistance base line.

### Example 21: Selectivity of Toluene MIP Sensor

By subjecting it to the other interfering gases, the selectivity of the MIP sensor to Toluene was studied. When exposed to Toluene, acetonitrile, acetone, and methanol, we assessed the sensor's reaction. The response values of Toluene and other interfering gases is found to be 8.42, 0.39, 0.33, and 0.58%. It can be observed that the sensor's response to all the interfering gases is negligible compared to that of 200 ppm of Toluene, as shown in the FIG. 13 a) and it depicts the selective behaviour of the sensor to Toluene.

### Example 22: Transient Studies of Toluene MIP Sensor

By subjecting it to same concentration of 1823 ppm, the Toluene MIP sensors were studied for its response and recovery for various cycles and found to exhibit same response level up to 4 cycle proving its repeatability and reproducibility of Toluene MIP sensor as shown in FIG. 13 b).

### Example 23: Imprinting Factor of Toluene MIP Sensor

The imprinting factor is the ratio of the response R(%) of the Toluene MIP and NIP and it is calculated to be 1.20. This proves that the Toluene VOC is imprinted perfectly during the polymerization reaction.

With its high sensitivity, the current Toluene MIP sensor offers significant potential for monitoring low quantities of Toluene in the biological systems and also in the environment. This Toluene MIP sensor marks as a significant step forward in the development of essential sensing materials for real-time volatile monitoring.

In an embodiment the aromatic compound can be selected from toluene, benzene, styrene, dimethylfuran, anthracene, dimethylnaphthalene, phenol, xylene, ethylbenzene, cyclohexene, trimethylbenzene, dichlorobenzene, hydroxytoluene, propylbenzene, phthalates, benzoates etc.

### Example 24: Preparation of MIP and NIP for Styrene (Eg 4-Vinylanisole)

4-Vinylanisole (4-VA) is an organic compound with a IUPAC name of 4-methoxy styrene. The fourth carbon atom of the aromatic ring has a vinyl group (CH₂═CH—) linked to it, making it a derivative of anisole (methoxybenzene). The most gregarious bug that generates vast swarms of insects and attacks the agriculture field is the migratory locust, Locusta migratoria. The pheromone emitted from their bodies attracts locusts. According to studies, 4-Vinylanisole attracts locusts strongly regardless of age or gender.

### Example 25: Preparation of 4-Vinylanisole MIP and NIP

The steps are as detailed in Example 1, changes if any are detailed here. The porogenic solvent combination is then ultrasonicated for about 15 minutes with 8-12 μL of 4-Vinylanisole (4-VA) as a template.

### Example 26: Plausible Mechanism for 4-Vinylanisole MIP Formation

The basic mechanism is detailed in Example 2. The mechanism and interaction between a functional monomer and template molecule (4-Vinylanisole) occur in the pre-polymerization process. 4-Methoxystyrene has the following resonance structures. The lone pair of electrons on methoxy oxygen are in resonance with the aromatic ring and therefore makes it less electronegative. But it can still form weak hydrogen bond complex with the carboxylic proton of MAA. Schematic representation of formation of molecularly imprinted polymers is provided in FIG. 1 and mechanism for the formation of MIP, NIP and removal of template in MIP is provided in FIG. 14.

### Example 27: FESEM Analysis of 4-Vinylanisole MIP

The FESEM micrographs of MIP coated PANI substrates (FIG. 15) before and after extraction reveals a change in their surface morphology. In FIG. 15 a) the macro porous structure of the MIP layer is visible as cracks at a 100 μm scale. When it is further magnified, aggregated spherical polymeric nanoparticles with smooth surface are visible at a 100 nm scale as in FIG. 15 b) on 4-Vinylanisole MIP before extraction. Similar to the FIG. 15 a) and b) the “after extraction sample” is found to have cracks with macro porous structure along with visible micro pores formed as voids after the removal of the template molecule as in FIG. 15 c). In a similar magnification of 100 nm scale as in FIG. 15 d) the aggregated spherical morphology found “before extraction sample” is changed into prominent spherical polymeric particles with rough surface sue to the extraction process.

### Example 28: Sensitivity of 4-Vinylanisole MIP Sensor

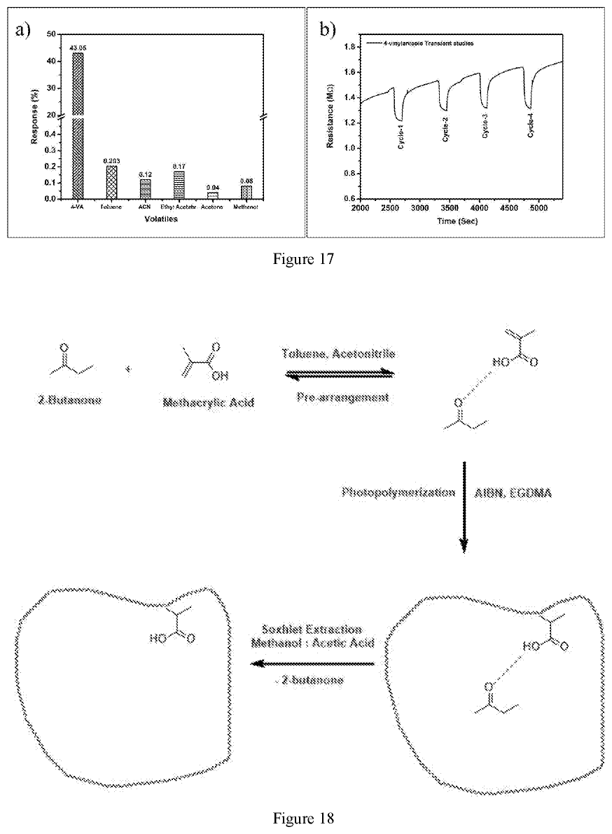

The response of 4-Vinylanisole to the MIP sensor were studied using their VOC chemiresistive sensing. The FIG. 16 a) displays the sensitivity of the MIP sensor for 4-Vinylanisole vapor maintained in an ice bath temperature at 4 °C with concentrations ranging from 2 ppm to 17 ppm. In FIG. 16 b) the response R(%) is calculated to obtain the response of the 4-Vinylanisole MIP sensor and it is calculated to be 17.63, 20.71, 28.56, 43.18 and 50.7% for 2 ppm, 4 ppm, 8 ppm, 12 ppm and 17 ppm. The sensor's high speed is confirmed by the typical response time of 32, 36, 22, 24, and 33 is observed with corresponding recovery time of 205, 228, 270, 401, and 533 seconds for 2 ppm, 4 ppm, 8 ppm, 12 ppm and 17 ppm as in FIG. 16 c). This explains the swiftness of the sensor in the low concentration as it responds faster. When the concentration is high the recovery time increases due to the unbinding of 4-Vinylanisole VOC from the binding sites of MIP. During sensing, the 4-Vinylanisole VOC binds with the active voids created due to the extraction. As a result of which a change in resistance value is observed with the corresponding concentration of the 4-Vinylanisole. We will be able to identify the traces of 4-Vinylanisole vapor in actual surroundings and precisely assess the low amounts. The 4-Vinylanisole MIP sensor exhibits a linear behaviour and exhibits a linear fit of R²=0.989 as shown in FIG. 16 d). The sensitivity of the 4-Vinylanisole sensor is measured to be 2.337*10⁻² ppm⁻¹. The theoretical lowest detection limit (LDL) of 4-Vinylanisole sensor is calculated to be 4 ppb. by measuring 100 points from the resistance base line.

### Example 29: Selectivity of 4-Vinylanisole MIP Sensor

By subjecting it to the other interfering gases, the selectivity of the MIP sensor to 4-Vinylanisole was studied. When exposed to 4-Vinylanisole, toluene, acetonitrile, ethyl acetate, acetone, and methanol, we assessed the sensor's reaction. The response values of 4-Vinylanisole and other interfering gases is found to be 43.05, 0.203, 0.12, 0.17, 0.04, and 0.08%. It can be observed that the sensor's response to all the interfering gases is negligible compared to that of 10 ppm of 4-Vinylanisole, as shown in the FIG. 17 a) and it depicts the selective behaviour of the sensor to 4-Vinylanisole.

### Example 30: Transient Studies of 4-Vinylanisole MIP Sensor

By subjecting it to same concentration of 8 ppm, the 4-Vinylanisole MIP sensors were studied for its response and recovery for various cycles and found to exhibit same response level up to 4 cycles proving its repeatability and reproducibility of 4-Vinylanisole MIP sensor as shown in FIG. 17 b).

### Example 31: Imprinting Factor of 4-Vinylanisole MIP Sensor

The imprinting factor is the ratio of the response R(%) of the 4-Vinylanisole MIP and NIP and it is calculated to be 1.16. This proves that the 4-Vinylanisole VOC is imprinted perfectly during the polymerization reaction.

With its high sensitivity, the current 4-Vinylanisole MIP sensor offers significant potential for monitoring low quantities of 4-Vinylanisole in the environment. This 4-Vinylanisole MIP sensor marks as a significant step forward in the development of essential sensing materials for real-time volatile monitoring.

In an embodiment the styrene can be selected from 4-vinylanisole, 4-methoxystyrene, vinylbenzene, ethenylbenzene, cinnamene, phenylethylene, etc.

### Example 32: Preparation of MIP and NIP for Ketone (Eg 2-Butanone)

The oxidation or dehydrogenation of 2-butanol can result in 2-Butanone, also known as methyl ethyl ketone. In GI Tract cancer patients, 2-Butanone is one of the main volatiles generated from precancerous lesions. Lung, ovarian, and liver cirrhosis cancer patients can all have 2-Butanone found in their exhaled breath. Even worse, it is carcinogenic and genotoxic, significantly endangering human health.

### Example 33: Preparation of 2-Butanone MIP and NIP

The steps are as detailed in Example 1, changes if any are detailed here. The porogenic solvent combination is then ultrasonicated for about 15 minutes with 9-13 μL of 2-Butanone as a template.

### Example 34: Plausible Mechanism for 2-Butanone MIP Formation

The basic mechanism is detailed in Example 2. The mechanism and interaction between a functional monomer and template molecule (2-Butanone) occur in the pre-polymerization process. The acidic carboxylic proton of MAA and carbonyl oxygen of 2-Butanone form hydrogen bond complex. Schematic representation of formation of molecularly imprinted polymers is provided in FIG. 1 and mechanism for the formation of MIP, NIP and removal of template in MIP is provided in FIG. 18.

### Example 35: FESEM Analysis of 2-Butanone MIP

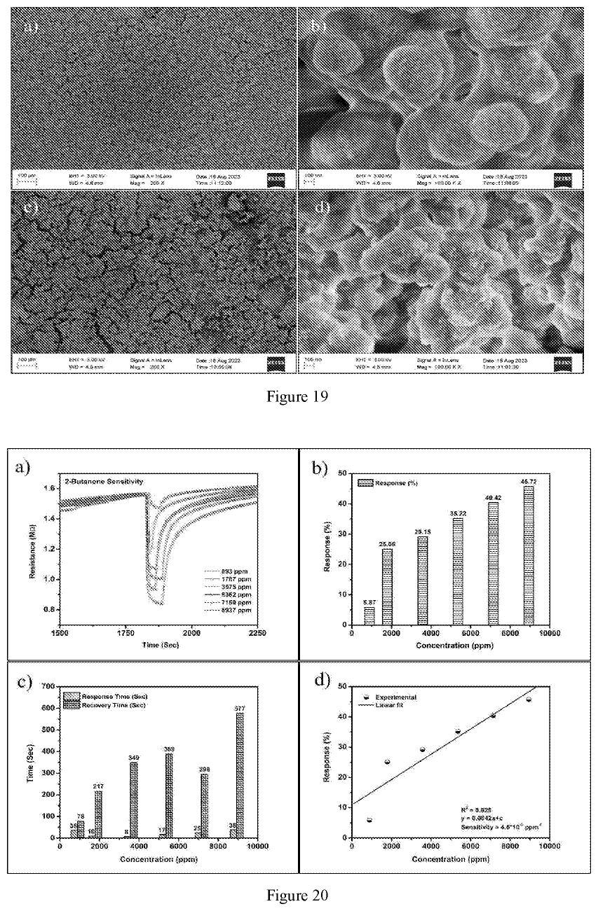

The FESEM micrographs of MIP coated PANI substrates (FIG. 19) before and after extraction reveals a change in their surface morphology. In FIG. 19 a) the macro porous structure of the MIP layer is visible as cracks at a 100 μm scale. When it is further magnified, uniform spherical polymeric nanoparticles are visible at a 100 nm scale as in FIG. 19 b) on 2-Butanone MIP before extraction. Similar to the FIG. 19 a) and b) the “after extraction sample” is found to have cracks with macro porous structure along with visible micro pores formed as voids after the removal of the template molecule as in FIG. 19 c). In a similar magnification of 100 nm scale as in FIG. 19 d) the spherical morphology found in “before extraction sample” is changed into irregular agglomerated particles.

### Example 36: Sensitivity of 2-Butanone MIP Sensor

The response of 2-Butanone to the MIP sensor were studied using their VOC chemiresistive sensing. The FIG. 20 a) displays the sensitivity of the MIP sensor for 2-Butanone vapor maintained in an ice bath temperature at 4 °C with concentrations ranging from 893 ppm to 8937 ppm. In FIG. 20 b) the response R(%) is calculated to obtain the response of the 2-Butanone MIP sensor and it is calculated to be 5.87, 25.06, 29.15, 35.22, 40.42, and 45.72% for 893 ppm, 1787 ppm, 3575 ppm, 5362 ppm, 7150 ppm and 8937 ppm. The sensor's high speed is confirmed by the typical response time of 36, 10, 8, 17, 25, and 38 seconds is observed with corresponding recovery time of 78, 217, 349, 389, 295, and 577 seconds for 893 ppm, 1787 ppm, 3575 ppm, 5362 ppm, 7150 ppm and 8937 ppm as in FIG. 20 c). This explains the swiftness of the sensor in the low concentration as it responds faster. When the concentration is high the recovery time increases due to the unbinding of 2-Butanone VOC from the binding sites of MIP. During sensing, the 2-Butanone VOC binds with the active voids created due to the extraction, as a result of which a change in resistance value is observed with the corresponding concentration of the 2-Butanone. We will be able to identify the traces of 2-Butanone vapor in actual surroundings and precisely assess the low amounts. The 2-Butanone MIP sensor exhibits a linear behaviour and exhibits a linear fit of R²=0.925 as shown in FIG. 20 d). The sensitivity of the 2-Butanone sensor is measured to be 4.2*10⁻⁵ ppm⁻¹. The theoretical lowest detection limit (LDL) of 2-Butanone sensor is calculated to be 6.2 ppm. by measuring 600 points from the resistance base line.

### Example 37: Selectivity of 2-Butanone MIP Sensor

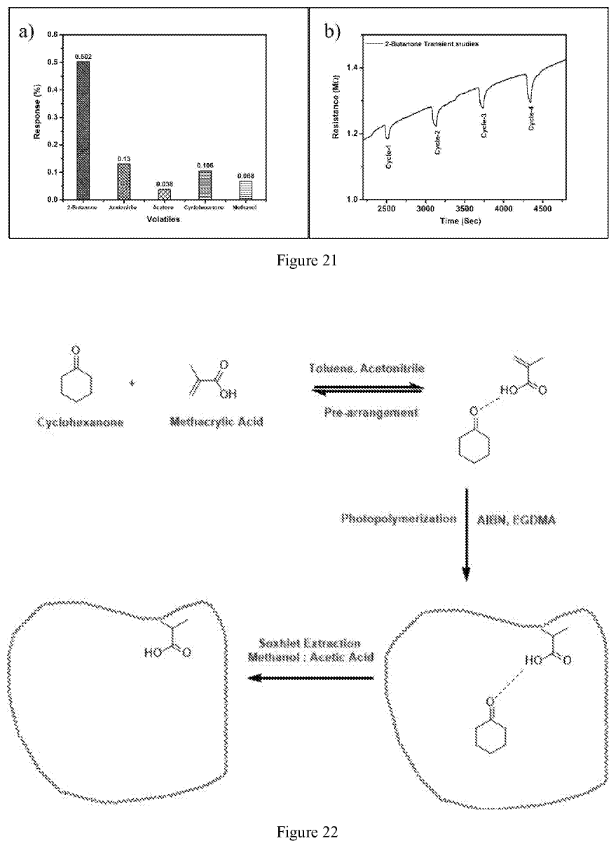

By subjecting it to the other interfering gases, the selectivity of the MIP sensors to 2-Butanone was studied. When exposed to 2-Butanone, acetonitrile, acetone, cyclohexanone, and methanol, we assessed the sensor's reaction. The response values of 2-Butanone and other interfering gases is found to be 0.502, 0.13, 0.038, 0.106, and 0.068%. It can be observed that the sensor's response to all the interfering gases is negligible compared to that of 10 ppm of 2-Butanone, as shown in the FIG. 21 a) and it depicts the selective behaviour of the sensor to 2-Butanone.

### Example 38: Transient Studies of 2-Butanone MIP Sensor

By subjecting it to same concentration of 3575 ppm, the 2-Butanone MIP sensors were studied for its response and recovery for various cycles and found to exhibit same response level up to 4 cycles proving its repeatability and reproducibility of 2-Butanone MIP sensor as shown in FIG. 21 b).

### Example 39: Imprinting Factor of 2-Butanone MIP Sensor

The imprinting factor is the ratio of the response R(%) of the 2-Butanone MIP and NIP and it is calculated to be 1.28. This proves that the 2-Butanone VOC is imprinted perfectly during the polymerization reaction.

With its high sensitivity, the current 2-Butanone MIP sensor offers significant potential for monitoring low quantities of 2-Butanone in the biological systems. This 2-Butanone MIP sensor marks as a significant step forward in the development of essential sensing materials for real-time volatile monitoring.

In an embodiment the ketone is selected from 2-butanone, pentenone, acetone, pentanone, hexanone, heptanone, benzophenone, hendecanone, pentadecanone, methyl isobutyl ketone, heptadecanone etc.

### Example 40: Preparation of MIP and NIP for Cyclicketone (Eg Cyclohexanone)

Cyclohexanone is an organic compound with six-carbon cyclic molecule with a ketone functional group. The inflammatory illness known as chronic obstructive pulmonary disease (COPD) is characterized by oxidative stress and the production of volatile organic compounds (VOCs) that are released via the lungs. A single analyte, cyclohexanone, has been found in a study using patients' exhaled breath with excellent sensitivity, specificity, and a positive predictive value of 95% for COPD. Also, Cyclotrimethylenetrinitramine (RDX) is one of the most often used energetic components in plastic explosive formulations. But, RDX has a low vapor pressure at room temperature, it can be sensed by detecting cyclohexanone, a non-explosive vapor that is left over when RDX recrystallizes.

### Example 41: Preparation of Cyclohexanone MIP and NIP

The steps are as detailed in Example 1, changes if any are detailed here. The porogenic solvent combination is ultrasonicated for about 15 minutes with 13-18 μL of cyclohexanone as a template.

### Example 42: Plausible Mechanism for Cyclohexanone MIP Formation

The basic mechanism is as detailed in Example 2. The mechanism and interaction between a functional monomer and template molecule (cyclohexanone) occur in the pre-polymerization process. Cyclohexanone is an aromatic ketone which can form hydrogen bond complex with the carboxylic acid proton of MAA. Schematic representation of formation of molecularly imprinted polymers is provided in FIG. 1 and mechanism for the formation of MIP, NIP and removal of template in MIP is provided in FIG. 22.

### Example 43: FESEM Analysis of Cyclohexanone MIP

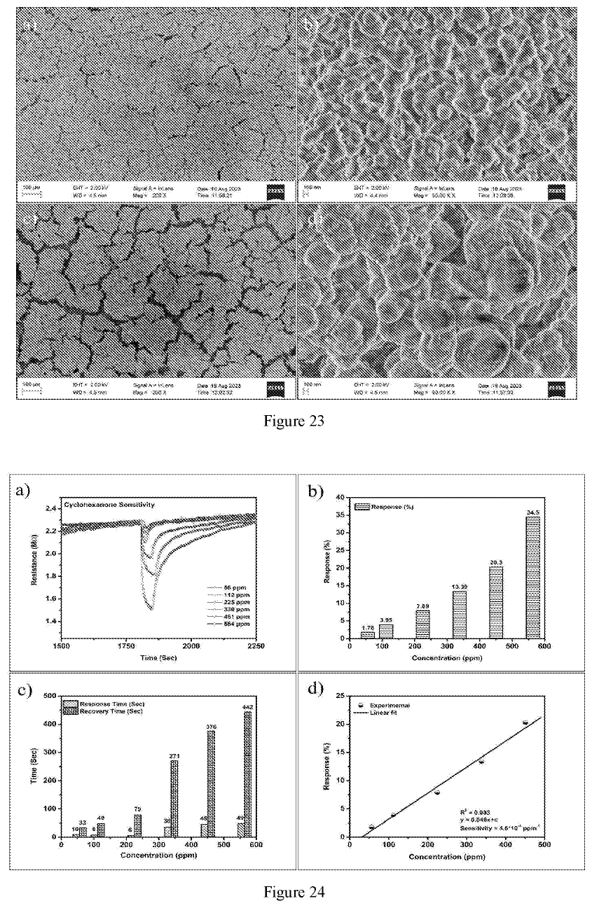

The FESEM micrographs of MIP coated PANI substrates (FIG. 23) before and after extraction reveals a change in their surface morphology. In FIG. 23 a) the macro porous structure of the MIP layer is visible as cracks at a 100 μm scale. When it is further magnified, uniform smaller spherical polymeric nanoparticles are visible at a 100 nm scale as in FIG. 23 b) on Cyclohexanone MIP before extraction. Similar to the FIG. 23 a) and b) the ‘“after extraction sample’” is found to have cracks with macro porous structure along with visible micro pores formed as voids after the removal of the template molecule as in FIG. 23 c). In a similar magnification of 100 nm scale as in FIG. 23 d) the spherical morphology found in “before extraction sample” is changed into irregular agglomerated particles with surface roughness.

### Example 44: Sensitivity of Cyclohexanone MIP Sensor

The response of Cyclohexanone to the MIP sensor were studied using their VOC chemiresistive sensing. The FIG. 24 a) displays the sensitivity of the MIP sensor for Cyclohexanone vapor maintained in an ice bath temperature at 4 °C with concentrations ranging from 56 ppm to 564 ppm. In FIG. 24 b) the response R(%) is calculated to obtain the response of the Cyclohexanone MIP sensor and it is calculated to be 1.76, 3.95, 7.89, 13.39, 20.3 and 34.5% corresponding to concentrations of 56 ppm, 112 ppm, 225 ppm, 338 ppm, 451 ppm and 564 ppm. The sensor's high speed is confirmed by the typical response time of 10, 8, 6, 36, 46, and 49 is observed with corresponding recovery time of 33, 48, 79, 271, 376 and 442 seconds for 56 ppm, 112 ppm, 225 ppm, 338 ppm, 451 ppm and 564 ppm as in FIG. 24 c). This explains the swiftness of the sensor in the low concentration as it responds faster. When the concentration is high the recovery time increases due to the unbinding of Cyclohexanone VOC from the binding sites of MIP. During sensing, the Cyclohexanone VOC binds with the active voids created due to the extraction, as a result of which a change in resistance value is observed with the corresponding concentration of the Cyclohexanone. We will be able to identify the traces of Cyclohexanone vapor in actual surroundings and precisely assess the low amounts. The Cyclohexanone MIP sensor exhibits a linear behaviour and exhibits a linear fit of R²=0.993 as shown in FIG. 24 d). The sensitivity of the Cyclohexanone sensor is measured to be 4.6*10⁻⁴ ppm⁻¹. The theoretical lowest detection limit (LDL) of Cyclohexanone sensor is calculated to be 2 ppm. by measuring 600 points from the resistance base line.

### Example 45: Selectivity of Cyclohexanone MIP Sensor

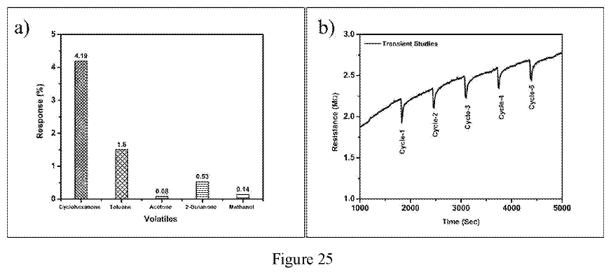

By subjecting it to the other interfering gases, the selectivity of the MIP sensor to Cyclohexanone was studied. When exposed to Cyclohexanone, toluene, acetone, 2-Butanone, and methanol, we assessed the sensor's reaction. The response values of Cyclohexanone and other interfering gases is found to be 4.19, 1.5, 0.08, 0.53, and 0.14%. It can be observed that the sensor's response to all the interfering gases is negligible compared to that of 50 ppm of Cyclohexanone, as shown in the FIG. 25 a) and it depicts the selective behaviour of the sensor to Cyclohexanone.

### Example 46: Transient Studies of Cyclohexanone MIP Sensor

By subjecting it to same concentration of 225 ppm, the Cyclohexanone MIP sensors were studied for its response and recovery for various cycles and found to exhibit same response level up to 5 cycles proving its repeatability and reproducibility of Cyclohexanone MIP sensor as shown in FIG. 25 b).

### Example 47: Imprinting Factor of Cyclohexanone MIP Sensor

The imprinting factor is the ratio of the response R(%) of the Cyclohexanone MIP and NIP and it is calculated to be 0.95. This proves that the Cyclohexanone VOC is imprinted during the polymerization reaction.

With its high sensitivity, the current Cyclohexanone MIP sensor offers significant potential for monitoring low quantities of Cyclohexanone in the biological systems and also in the environment. This Cyclohexanone MIP sensor marks as a significant step forward in the development of essential sensing materials for real-time volatile monitoring.

In an embodiment the cyclic ketone can be selected from cyclohexanone, cyclopentanone, cycloheptanone, cyclooctenone.

The MIP sensor prepared from the combinational technique of bulk and photopolymerization has various advantages such as small solvent volume, less reaction time, structural stability, easy removal of template and precise binding sites for template volatile organic compounds. Compared to MIP in powder forms, the prepared sensor has lower interparticle distance which enhances the sensing properties. MIP sensor are for the detection of any VOCs (not limited to plant, human, microbe, pollutants, drugs, contrabands and explosives) with high sensitivity and superior selectivity.

It should be understood that the examples described herein are for illustrative purposes only and that various modifications or changes in light if the specification will be suggestive to person skilled in the art and are to be included within the purview and scope of this patent.

1. A method of preparing molecular imprinted polymer over a semiconducting layer coated substrate, comprising steps of:
preparing a porogen solvent in an ratio of 6.75 and 0.75 mL v/v, in a glass container;
sonicate the porogen solvent for about 30-90 seconds;
add a mixture containing template volatile organic compound and about 20-30 μL monomer into the glass container and sonicate the contents for about 15 minutes;
add about 110-120 μL cross-linker and purge the glass container with nitrogen gas for about 120 seconds, the ratio of template volatile organic compound, monomer, and cross-linker are kept constant at as n:4:8, wherein ‘n’ ranges about 1-5;
sonicate the contents for about 10 minutes;
stir the contents for about 10 minutes;
about 10-15 mg of initiator is added to the above content and stirred for about 15 minutes in a nitrogen atmosphere, to obtain a pre polymer mixture;
semiconducting layer coated substrate is immersed in the pre polymer mixture and purged with nitrogen for about 2 minutes before carefully sealing the glass container;
the sealed glass container is placed in a UV chamber and photopolymerization is carried out at room temperature at wavelength (λ) of about 350 nm for about 60-100 min using a 8 W UV bulb;
the semiconducting layer coated substrate were removed after about 6-10 min and dried in normal atmospheric conditions;
a thick white layer of molecularly imprinted polymer forms on the surface of the semiconducting layer coated substrate in about 5-10 seconds;
the molecularly imprinted polymer-semiconducting layer coated substrates are immersed in a mixture of about 20-50 μL acetic acid and about 150-200 mL methanol solution, and extracted for about 12 hours using a soxhlet apparatus at about 80-90 °C for the removal of template volatile organic compounds and creation of template-voids;
to obtain molecular imprinted over semiconducting layer coated substrate;
wherein the porogen solvent is acetonitrile and toluene, 2-methoxyethanol, methanol, chloroform, tetrahydrofuran (THF), dichloroethane, and ionic liquids as 1-Butyl-3 methylimidazolium hexafluorophosphate [BMIM][PF6], 1-Butyl-3 methylimidazolium tetrafluoroborate [BMIM][BF4], 1-Methyl-3-octyl-imidazolium-hexafluorophosphate [OMIM][PF6], 1-Hexyl-3-methyl-imidazolium-hexafluorophosphate [HMIM][PF6],
wherein the substrate is selected from ceramic alumina, glass slides, fabrics, fluorine doped tin oxide substrates, indium doped tin oxide substrates, flexible polymer substrates.

2. The method of claim 1, wherein the template volatile organic compound is selected from alcohol, aldehyde, aromatic compounds, styrene, ketone, cyclic ketone, esters, amines, organochlorides, organophosphates, alkanes, alkyne, alkenes.

3. The method of claim 1, wherein,
the alcohol is selected from 2-phenylethanol, hexenol, linalool, nerolidol, ethanol, methanol, pinocarveol, isopropyl alcohol, pentanol;
the aldehyde is selected from heptanal, nonanal, pentanal, hexanal, acetaldehyde, formaldehyde, butanal, octanal, octenal, benzaldehyde, phenylacetaldehyde, cinnamaldehyde, cuminaldehyde, (E,E)-α-farnesene-2(3),9(10)-diepoxide, malondialdehyde, hydroxyacetalydehyde, 2-butenal, dimethylbenzaldehyde, phenylpropionaldehyde, perillic aldehyde, methional, hexenal, pentenal;
the aromatic compound is selected from toluene, benzene, styrene, dimethylfuran, anthracene, dimethylnaphthalene, phenol, xylene, ethylbenzene, cyclohexene, trimethylbenzene, dichlorobenzene, hydroxytoluene, propylbenzene, phthalates, benzoates;
the styrene is selected from 4-vinylanisole, 4-methoxystyrene, vinylbenzene, ethenylbenzene, cinnamene, phenylethylene;
the ketone is selected from 2-butanone, pentenone, acetone, pentanone, hexanone, heptanone, benzophenone, hendecanone, pentadecanone, methyl isobutyl ketone, heptadecanone; and
the cyclic ketone is selected from cyclohexanone, cyclopentanone, cycloheptanone, cyclooctenone.

4. The method of claim 1, wherein the semiconducting layer is selected from polyaniline, carbon nantotubes, activated carbon, graphene oxide, reduced graphene oxide, class of MXene materials, siloxene, phosphorene, black phosphorus germanene and graphitic carbon nitride, metal oxides, metal chalcogenides, metal phosphides, metal vanadates, etc their combinations of composite materials and alloys thereof.

5. The method of claim 1, wherein the porogen solvent is acetonitrile and toluene.

6. The method of claim 1, wherein the monomer is selected from methacrylic acid, acrylamide, 4-Vinylpyridine, acrylic acid, 2-Hydroxyethyl Methacrylate (HEMA), styrene, N, N′-Methylenebisacrylamide (BIS), N-Methacryloyl-(L)-phenylalanine (MAPA), N-Isopropylacrylamide (NIPAM), divinylbenzene (DVB).

7. The method of claim 1, the crosslinker is selected from ethylene glycol dimethacrylate, trimethylolpropane trimethacrylate (TRIM), pentaerythritol tetraacrylate (PETA), N,N′-Methylenebisacrylamide (BIS), 1,4-Butanediol Dimethacrylate (BDDMA), 1,2-Bis(allyloxy)ethane (BAE).

8. The method of claim 1, the initiator is selected from 2,2′-azobisisobutyronitrile (AIBN), 2,2-dimethoxy-2-phenylacetophenone (DMPA), azobis (2-methylpropionitrile) (AMBN), Benzoyl peroxide, 2,2′-azobis(2,4-dimethylvaleronitrile) (ADMVN), N,N,N′,N′-tetramethylethylenediamine (TEMED).

9. The method of claim 1, wherein the polyaniline coated substrate for molecular imprinting is prepared by the steps of:
preparing a cationic precursor solution containing about 0.1M aniline in about 0.1M sulphuric acid;
preparing an anionic precursor solution containing about 0.1M ammonium persulphate in 0.1M of sulphuric acid and anionic precursor solution is maintained at 4 °C;
substrates were first immersed in the cationic precursor for about 20 seconds,
secondly, substrates were immersed in the anionic precursor for about 15 seconds,
continuing the immersion process in the cationic and the anionic precursor solutions for about 20 seconds and about 15 seconds alternately for about 50 cycles,
to obtain polyaniline coated substrates;
further, the polyaniline coated substrate is immersed in double distilled water to remove unbound and unreacted anilinium ions; and
the polyaniline coated substrate is dried in vacuum oven at about 80-90 °C for about 24 hours.

10. The method of claim 1, wherein the semiconducting layer coated substrate for molecular imprinting is prepared by UV photopolymerization, bulk polymerization, electro-polymerization, microwave polymerization, precipitation polymerization, Reversible addition fragmentation chain-transfer (RAFT) polymerization, surface imprinting, lithography imprinting, immobilization imprinting, micro contact imprinting, and emulsion polymerization.

11. Molecular imprinted polyaniline coated ceramic substrate obtained by the method of claim 1.

12. A sensor, for sensing volatile organic compounds, comprising:
at least two electrodes, and
a molecularly imprinted polymer over the semiconducting layer coated substrate of claim 1 as sensing element, electrically connected to the two electrodes.

13. The sensor of claim 12, wherein the distribution of the molecularly imprinted polymer over semiconducting layer is in the form of a continuous layer with template-voids, wherein the entire layer is electrically conductive.

14. The sensor of claim 12, wherein analysing the values generated by the sensor comprising a resistance change including changes in, capacitance, voltage threshold, frequency, inductance, impedance, conductance, mobility, optical property or any combination thereof.
